(19) United States
(12) Patent Application Publication
(10) Pub. No.: US 2005/0074742 A1
(43) Pub. Date: Apr. 7, 2005

(54) Cartilaginous neo-tissue capable of being grafted

(76) Inventors: Alain Domard, Lyon (FR); Marie-Therese M. Corvol, Paris (FR); Francois Guillot, Antibes (FR); Xavier L. Chevalier, Paris (FR); Isabelle Morfin, Grenoble (FR); Dominique J. Vacher, Mougin (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/473,359

(22) PCT Filed: Mar. 29, 2002
(86) PCT No.: PCT/FR02/01122

(30) Foreign Application Priority Data
Mar. 30, 2001 (FR) 01/04405
Aug. 31, 2001 (FR) 01/11322

(51) Int. Cl. C12N 5/00; C12N 5/02

(52) U.S. Cl. 435/1.1; 435/395

(57) ABSTRACT

A cartilaginous neo-tissue that is capable of being grafted is constituted by rows of approximately parallel cells with a cell maturation gradient orientated from a predetermined zone towards its periphery. During preparation of the cartilaginous neo-tissue with a chitosan hydrogel, the predetermined zone corresponds to the junction of cells with the chitosan hydrogel in contact with which the neo-tissue develops.

The present invention relates to the field of repairing cartilaginous lesions by means of a graft. More particularly, it relates to a method of preparing cartilaginous neo-tissue that is capable of being grafted.

### BACKGROUND OF THE INVENTION

Cartilage is a tissue of mesenchymal origin constituted by a small percentage of chondrocytes distributed in an extracellular matrix which is renewed by them. That matrix is composed of a network of collagen fibers, in particular type II fibers, and glycosaminoglycans associated with structure proteins to form proteoglycans. The amphiphilic nature and ionic sites of the ensemble produce a physical gel ensuring the viscoelastic properties of the cartilaginous tissue.

Cartilaginous tissue disappears in adulthood apart from at the joints, where it ensures that articular surfaces can move and tolerate large compressive loads. However, articular cartilage is not capable of spontaneous regeneration. For that reason, graft techniques such as mosaic grafts or autologous cell grafts are used in the event of cartilaginous lesions.

Mosaic grafts consist of removing bone covered in cartilage from non bearing regions and grafting them into the lesion.

Autologous cell grafts consist of removing healthy cartilage, carrying out enzymatic digestion to release chondrocytes from the extracellular matrix and multiplying the chondrocytes ex vivo to obtain a sufficient number of chondrocytes, which are then re-implanted into the cartilaginous lesion. Since the chondrocytes are in the form of a cell suspension in an aqueous medium (dispersion in a liquid medium), the excised lesion must first be covered with a membrane formed from periosteum securely sutured to the edge of the cartilage, then the chondrocyte suspension (dispersion containing the culture) is injected into the cavity that is created. After a certain period, those cells produce an extracellular matrix which, however, does not have the tissue organization of normal articular cartilage.

It should be noted that the mode of multiplication of the chondrocytes to be implanted must be determined so as to avoid cell dedifferentiation. In particular, if chondrocytes are proliferated on a support (synthetic polymer) such as the bottom of cell culture dishes, chondrocytes dedifferentiate into fibroblast cells. They are then fusiform instead of being polygonal, like chondrocytes, and synthesize collagen I instead of collagen II.

International patent document WO-A-00/56251 proposes multiplying cells, including human chondrocytes, on biodegradable polysaccharide beads cross-linked by polyamines. The polysaccharides are selected from the following compounds: dextran, cellulose, arabinogalactan, pullulan, and amylase. The cross-linking agent is glutamic acid, lysine, albumin or gelatin, for example.

According to that document, after bringing the chondrocytes into contact with said polymer beads with mechanical agitation, the chondrocytes multiply, retaining their form and phenotype; more particularly, they synthesize collagen II.

After said multiplication, the chondrocytes are recovered by digesting the polysaccharide beads using specific enzymes, for example dextranase, which does not alter chondrocyte cells.

Those cells are then detached for inclusion into a chitosan matrix. To this end, chondrocytes are added to an acid chitosan solution, then the mixture is agitated until a three-dimensional structure is formed, which is placed in a 1N sodium hydroxide solution to precipitate out the chitosan over several minutes. After polymerization, the sodium hydroxide is rapidly eliminated, then the polymerized conglomerate of chitosan and cells is cultured at 37° C. under 5% CO₂ for a predetermined period.

Thus, according to WO-A-00/56251, the chondrocytes mixed with the chitosan are incorporated into the three-dimensional structure of precipitated chitosan, which structure should have a firm consistency resembling the texture of cartilage.

WO-A-00/56251 describes a further possible variation in the first chondrocyte multiplication step, namely multiplying said cells on a chitosan film. The two other steps remain the same; the second step consists in extracting the multiplied chondrocytes by enzymatic digestion using collagenase or trypsin and the third step consists in including said chondrocytes in a three-dimensional chitosan matrix under the conditions described above.

Chitosan is obtained by deacetylating chitin, the most common biopolymer to be found in nature after cellulose. Chitin can be extracted from the exoskeleton of certain crustaceans such as the lobster or crab, or from the squid endoskeleton, for example. Chitin and chitosan are constituted by the same two monomer units, N-acetyl-D-glucosamine and D-glucosamine. When the polymer is highly acetylated, i.e. when it comprises more than 60% of N-acetyl-D-glucosamine, it is known as chitin. Both are biodegradable, bioresorbable and compatible with living tissue.

Chitosan is known to have a biostimulating activity on tissue reconstitution. However, it is generally used in association with other elements. As an example, in WO-A-96/02259, chitosan is combined with another polysaccharide to form an agent for stimulating and regenerating hard tissue at an integration site for an implant, for example a titanium implant.

In WO-A-99/47186, for example, chitosan is cross-linked with glycosaminoglycan to constitute a biochemical environment that is close to cartilaginous tissue, stimulating cell growth.

The methods described in WO-A-00/56521 and WO-A-99/47186 are based on the “scaffold” technique in which the cells which are incorporated and included in a three-dimensional structure which forms a scaffold or framework. Said three-dimensional structure, constituted by chitosan alone in WO-A-00/56521 or associated with other constituents in WO-A-99/47186, forms an integral part of the material intended to be grafted.

### OBJECTS AND SUMMARY OF THE INVENTION

The cartilaginous neo-tissue that is capable of being grafted of the present invention differs from the disclosure of the prior art documents in that it does not comprise a component forming a three-dimensional scaffold type structure.

In accordance with the present invention, said cartilaginous neo-tissue is constituted by rows of approximately parallel cells with a cell maturation gradient orientated from a predetermined zone towards its periphery.

In particular, said cartilaginous neo-tissue is obtained by a method consisting in:
a) culturing chondrogenic cells, which are either autologous chondrocytes or chondrocyte precursor cells prepared in vitro from pluripotent stem cells;
b) bringing said chondrogenic cells into contact with a chitosan hydrogel having amphiphilic properties and a degree of acetylation such that said cells adhere naturally to the outer surface of said hydrogel;
c) covering the hydrogel/cell ensemble obtained with a culture medium; and
d) allowing a cartilaginous neo-tissue to develop in contact with the chitosan hydrogel for a minimum period of two weeks, frequently renewing the culture medium.

Thus, in contrast to that which is proposed in WO-A-00/56251, the chondrogenic cell amplification method is carried out either spontaneously in the presence of the chitosan hydrogel, or after prior amplification under conventional high density culture conditions, and the extra-cellular matrix is formed simultaneously in the presence of the chitosan hydrogel.

The natural adhesion of cells to the outer surface of the chitosan hydrogel can produce very good distribution of said cells and prevents the loss of cells during the operation, for example when it is carried out in culture wells.

The chitosan hydrogel acts as an inducer on the chondrogenic cell phenotype, which proliferate without dedifferentiating.

It should be noted that the chondrogenic cells do not penetrate directly into the hydrogel, which has a pore size that is insufficient compared with the size of said cells. The chitosan hydrogel is progressively metabolized and/or replaced and/or invaded by cartilage type matrix proteins, which are neo-synthesized by the chondrocytes. After at least two weeks of culture, the ensemble produces cartilaginous neo-tissue which can be grafted as is; the chitosan hydrogel, which serves as a temporary support for said cartilaginous neo-tissue, is partially or completely biodegraded.

The degree of acetylation of the chitosan used to prepare the hydrogel is in the range 30% to 70%, preferably in the range 40% to 60%.

In a first variation, the chondrogenic cells are brought into contact with the outer surface of the chitosan hydrogel which is in the form of small particles with a size of several millimetres.

In a second variation, the chondrogenic cells are spread in the form of at least one sheet between at least two layers of chitosan hydrogel, each layer being of the order of a few millimeters thick. This particular disposition can very readily produce cartilaginous neo-tissue of large size after complete disappearance of the chitosan hydrogel.

The cartilage neo-tissue formed using the method of the invention is constituted by rows of approximately parallel cells, with a cell maturation gradient orientated from a predetermined zone to its periphery, the predetermined zone corresponding to the junction of the cells with the chitosan hydrogel. When said neo-tissue is analyzed histologically, its morphological appearance is close to that of normal cartilaginous tissue.

### DETAILED DESCRIPTION OF THE INVENTION

The present invention will be better understood from the following description, made with reference to some examples describing the preparation of cartilaginous neo-tissue using a chitosan hydrogel with a degree of acetylation in the range 40% to 60% as the amplification support.

Purification of Chitosan

The reference chitosan used was obtained from squid endoskeletons. It had a degree of acetylation of 5.2%. It was initially purified to eliminate insoluble particles by carrying out the following steps: dissolving, filtering, precipitating, washing, and freeze drying.

For dissolution, a low viscosity solution was prepared with a concentration of the order of 0.5% by weight of chitosan in an acid solution. More precisely, acetic acid was added in stoichiometric quantities with respect to the amine groups of the chitosan.

The polymer solution was filtered by successive passes over membranes with decreasing pore sizes (1.2 micrometers (μm); 0.8 μm and 0.45 μm) under a maximum pressure of 3 bars.

The polymer was precipitated by increasing the pH of the solution by adding concentrated ammonia solution, with agitation.

Several washing operations were then required to reduce the pH of the suspension by eliminating excess ammonia. After each wash, the suspension was centrifuged and the residue was recovered. Washing was continued until the pH of the wash water was constant at a value which depended on the degree of acetylation.

Freeze drying produced solid chitosan.

Chitosan Acetylation

The chitosan was then re-acetylated to obtain the desired degree of acetylation. Said re-acetylation was carried out by reacting the amine function with acetic is anhydride in a hydro-alcoholic medium. The ratio between the number of amine functions and the number of anhydride molecules present in solution determined the degree of acetylation of the chitosan produced. For a chitosan with a given degree of acetylation, a hydro-alcoholic solution was used which, in addition to the chitosan, comprised water, 1,2-propanediol and the quantity of acetic acid required to produce stoichiometric proportions with respect to the amine functions of the chitosan. In a more precise example, the hydro-alcoholic solution comprised 3 grams (g) of chitosan, 323 g of water and 272 g of 1,2-propanediol. The acetylating mixture comprised acetic anhydride and propanediol. For 62.38 g of propanediol, for example, the mixture comprised 1.26 milliliters (ml) of acetic anhydride to obtain a degree of chitosan acetylation of 50% and 1.62 ml of acetic anhydride to obtain a degree of acetlylation of 60%.

Formation of a Physical Chitosan Hydrogel

Said formation necessitated passing from a liquid state to a gel state. Said passage corresponded to an initial situation (liquid state) in which hydrophilic interactions dominated to a final situation (hydrogel state) in which hydrophobic interactions became sufficiently strong for there to be no more dissolution without, however, being strong enough to cause complete precipitation of the polymer. The preferred preparation mode, in accordance with the invention, was to start from an initial solution of chitosan. If necessary, depending on the degree of acetylation, the solution could be acidic with the chitosan being dissolved in hydrochloric acid in stoichiometric quantities with respect to the amine groups of the chitosan. After completely dissolving the chitosan, a certain volume of 1,2-propanediol was added dropwise to the solution which was then vacuum degassed for a period of about one hour. The solution was then poured into a receptacle that provided a large free surface/volume ratio and was placed in an oven at 45° C. for the time required for the gel to set. The chitosan hydrogel was thus produced by a physico-chemical method.

To obtain a hydrogel which was not soluble in water at pHs of the order of 6 or 7, the hydrogel obtained was neutralized by placing it for about one hour in a basic medium, for example 0.1 molar sodium hydroxide.

The reduction in the number of positive charges due to the pH increase enhanced hydrophobic interactions and thus enhanced the stability of the gel. The hydrogel was then washed to eliminate the alcohol and obtain a pH of about 7. That washed chitosan hydrogel was used to culture chondrogenic cells.

It should be noted that gel setting corresponded to a predetermined aqueous solution/1,2-propanediol ratio, which ratio depended on the degree of acetylation of the chitosan. Further, since gelling is accompanied by a loss of water, the operating conditions had to encourage said water evaporation.

A number of types of receptacles could be used during gelling, either Petri dishes, multi-well plates or inserts specially designed to be housed in the wells of multi-well plates. As an example, a plate of 24 wells could be provided with inserts, each insert being constituted by a plastic cone the base of which was formed from a membrane that was permeable to the nutrient liquid and arranged to be placed in each well without touching the bottom.

Culture

The chondrogenic cells could be autologous chondrocytes or precursor chondrocyte cells prepared in vitro from pluripotent stem cells.

Regardless of the receptacle used to form it, the hydrogel obtained was in the form of a viscoelastic, translucent block the capacity and strength of which depended in particular on the concentration of chitosan in the initial solution. Preferably, said concentration was 0.5% to 4%. In order to culture chondrogenic cells, it was first necessary to increase the contact surface area between the chitosan hydrogel and said cells. To this end, in a first variation, the chitosan hydrogel block was cut into small fragments with external dimensions of the order of a few millimeters. Said fragments were disposed in the wells of a multi-well plate or, possibly, in inserts provided in said plate. The chondrogenic cells were introduced in the form of a suspension and carefully mixed with the hydrogel fragments. The ensemble was covered with a suitable culture medium. It was determined that the chondrogenic cells adhered spontaneously to the outer surface of the hydrogel fragments and did not drop into the well bottom. Culture was carried out by placing the filled plates in an atmosphere of 10% CO₂ at 37° C. The nutrient medium was renewed twice a week. Culture was continued for a period of 2 to 6 weeks depending on the desired size of the cartilaginous neo-tissue which formed in contact with the chitosan hydrogel.

A “number of cells/chitosan hydrogel fragment” proportion or ratio had to be selected to prevent, as far as possible, certain cells from falling into the well bottom. In one example, 5×10 chondrogenic cells were placed per thirty (approximately) chitosan hydrogel fragments per insert, or approximately 1 to 3×10⁶ chondrogenic cells per hundred chitosan hydrogel fragments per well not provided with an insert.

The degree of acetylation, in the range 30% to 70%, but preferably in the range 40% to 60%, induced optimum amphiphilic conditions that encouraged the establishment of an environment propitious to the synthesis of cartilaginous neo-tissue. By increasing the degree of acetylation, hydrophobic interactions due to the N-acetamide functions were increased. Simultaneously, the cationic nature of the residual amine sites was also increased, thereby reinforcing their hydrophilic nature and their ability to create electrostatic interactions. All said conditions were favorable to establishing interactions with the proteoglycans of the extracellular matrix neo-formed by the chondrogenic cells.

Further, the pH conditions, of the order of 7, were favorable to the action of enzymes, for example lysosyme, secreted by the chondrocytes and allowing degradation by hydrolysis of the glycosuric bonds constituting the chitosan chain.

Under the conditions indicated above, the chondrocytes multiplied and simultaneously synthesized a substantial matrix which accumulated around the cells and replaced or progressively covered the chitosan hydrogel. It was also possible to follow the formation of said cartilaginous neo-tissue as a function of culture time. At the early culture stage, the chondrogenic cells adhered to the hydrogel without ever penetrating it; they secreted matrix proteins of the collagen and proteoglycan type which accumulated around the cells to form a layer that was more dense along the hydrogel between the cells and the hydrogel, which retained its initial appearance. When culture was continued, in particular over four to six weeks, the chondrogenic cells multiplied from cells on the edge of the hydrogel and matrix proteins continued to accumulate. The structure of the hydrogel was modified, becoming looser, progressively taking on colorations specific to collagen proteins and to proteoglycans. When culture was complete, a block of neoformed tissue was obtained constituted by a plurality of colonies of cells organized in approximately parallel rows and with a cell maturation gradient orientated from the junction of the cells with the hydrogel towards its periphery. By analyzing this block of neo-tissue histologically, it could be seen that its morphological appearance was close to that of a normal cartilaginous tissue. Molecular analysis using RT-PCR was carried out after five weeks of culture, for the expression of collagens I and II, agrecan, biglycan, and decorin. Messenger RNAs for collagen II, agrecan, biglycan, and decorin were expressed while those for collagen I were not detectable. On the protein level, proteoglycan synthesis was studied after incorporating sulfur 35. The proteoglycans were extracted from the neo-tissue using 4M guanidine chloride, purified, and then analyzed by sepharose 2B column chromatography. The elution profiles obtained showed that the cells had synthesized and secreted proteoglycans which were collected in the matrix in the form of high molecular weight aggregates of profile similar to those synthesized and secreted in vivo.

In a second variation, the chondrogenic cells were spread in the form of a sheet between layers of hydrogel, each layer having a thickness of the order of a few millimeters. As an example, four sheets of cells were spread in combination with three layers of hydrogel, namely two sheets respectively on the outer faces of the first and third layer of hydrogel and two sheets sandwiched respectively between the first and second layer and the second and third layer of hydrogel.

The cells were cultured under the same conditions as those described above and the same observations were made regarding the formation of cartilaginous neo-tissue. The cell colonies which were formed either from cells in contact with the outer face of the first and the third layer of hydrogel or from cells spread between two layers of hydrogel all had a morphological gradient similar to that described above. The hydrogel layers intercalated between the sheets of cells had disappeared and were replaced by a highly alcyanophilic fibril structure the thickness of which approximately corresponded to superimposing two or three layers of cells. It can be seen that this latter variation readily allows layers of chitosan hydrogel and chondrogenic cells to be superimposed to produce a larger size cartilaginous neo-tissue.

Regardless of the variation employed, the cartilaginous neo-tissue obtained using the method of the invention is capable of being grafted as is to repair cartilaginous or meniscal lesions or intervertebral disks, in particular large lesions.

1. A cartilaginous neo-tissue that is capable of being grafted, the neo-tissue being constituted by rows of approximately parallel cells with a cell maturation gradient orientated from a predetermined zone towards its periphery.

2. A neo-tissue according to claim 1, wherein the predetermined zone corresponds to the junction of cells with a chitosan hydrogel in contact with which the neo-tissue develops during its preparation.
